(19) United States
(12) Patent Application Publication
(10) Pub. No.: US 2020/0347249 A1
(43) Pub. Date: Nov. 5, 2020

(54) RESIN MOLDING FOR MEDICAL DEVICE AND MEDICAL DEVICE

(71) Applicant: OLYMPUS CORPORATION, Tokyo (JP)

(72) Inventor: Takashi MAGARA, Tokyo (JP)

(73) Assignee: OLYMPUS CORPORATION, Tokyo (JP)

(21) Appl. No.: 16/933,893

(22) Filed: Jul. 20, 2020

Related U.S. Application Data
(63) Continuation of application No. PCT/JP2018/037233, filed on Oct. 4, 2018.

(30) Foreign Application Priority Data
Jan. 30, 2018 (JP) 2018-013615

(51) Int. Cl. C09D 11/101; C09D 11/30; A61B 1/00; B29C 45/00; G09F 23/00

(52) U.S. Cl. CPC C09D 11/101; C09D 11/30; A61B 1/0011; A61B 2090/0807; B29C 45/0053; G09F 23/00; B29C 45/0001

(57) ABSTRACT

A resin molding for a medical device has a base material made of resin, and an indicator including a cured product of UV-curable ink. An uneven structure is formed on at least a part of the surface of the base material. The uneven structure includes a plurality of recesses. The plurality of recesses each have a width of 10 μm or more and 60 μm or less. The indicator is formed on the uneven structure.

### CROSS REFERENCE TO RELATED APPLICATIONS

This application is a continuation application based on a PCT Patent Application No. PCT/JP2018/037233, filed on Oct. 4, 2018, whose priority is claimed on Japanese Patent Application No. 2018-013615, filed on Jan. 30, 2018. The contents of both the PCT Application and the Japanese Application are incorporated herein by reference.

### BACKGROUND

### Technical Field

The present invention relates to a resin molding for a medical device and a medical device.

### Background Art

Medical devices such as endoscopes, for example, are subjected to a sterilization process. For example, various indicators are printed on the operation portion and the like of the endoscope. Examples of indicators include product logos, the model name, symbols for guiding operation, and the like. The indicators need to have enough durability not to disappear during the period of use. In particular, there is a demand for indicators for medical devices to have high adhesion able to withstand repeated sterilization processes. There is also a demand for indicators for medical devices to have chemical resistance able to withstand repeated sterilization processes.

Thermosetting ink is often used for indicators for medical devices. The thermosetting ink is printed on the surface of the medical device by, for example, screen printing, pad printing, or the like. The thermosetting ink is heated after printing and cured.

However, in screen printing and pad printing, it is necessary to prepare a printing plate for each indicator. Medical devices are often produced in various types and in small quantities. The operation of changing the indicator plates for each model causes an increase in manufacturing time.

As a printing method, ink jet printing is also known. In ink jet printing, it is not necessary to make a plate. For example, Japanese Unexamined Patent Application, First Publication No. 2015-163701 describes a technique relating to ink jet printing using an ultraviolet (UV) curable ink.

However, the background art described above has the following problems.

The UV-curable ink described in Japanese Unexamined Patent Application, First Publication No. 2015-163701 has lower adhesion to a printing target than a thermosetting ink used for a medical device. When the adhesion to the printing target is low, the adhesive is easily peeled off from the printing target. Even in a case where the material of the UV-curable ink itself has chemical resistance, the UV-curable ink is easily peeled off from the printing target in the medical device due to wear and tear caused by repeated sterilization processes.

As described above, it is difficult to use an ink jet-printable UV-curable ink for indicators for a medical device to be subjected to sterilization processes.

The present invention was made in view of the problem described above and has an object of providing a resin molding for a medical device, which is able to be manufactured quickly and easily and which is able to improve adhesion between an indicator and a base material, and a medical device.

### SUMMARY

A resin molding for a medical device of a first aspect of the present invention includes a base material made of resin, and an indicator including a cured product of UV-curable ink, wherein an uneven structure is formed on at least a part of a surface of the base material, the uneven structure includes a plurality of recesses, the plurality of recesses each have a width of 10 μm or more and 60 μm or less, and the indicator is formed on the uneven structure.

According to a resin molding for a medical device of a second aspect of the present invention, in the first aspect described above, a depth of each of the plurality of recesses may be 5 μm or more and 60 μm or less.

According to a resin molding for a medical device of a third aspect of the present invention, in the first aspect described above, an interval between recesses adjacent to each other among the plurality of recesses may be 1 μm or more and 30 μm or less.

According to a resin molding for a medical device of a fourth aspect of the present invention, in the first aspect described above, an arrangement of the plurality of recesses may be periodic.

According to a resin molding for a medical device of a fifth aspect of the present invention, in the first aspect described above, the plurality of recesses may include at least one of holes and grooves.

A medical device according to a sixth aspect of the present invention includes the resin molding for a medical device according to the first aspect.

The resin moldings for a medical device of the first to fifth aspects and the medical device of the sixth aspect described above are able to be manufactured quickly and easily, and are able to improve adhesion between an indicator and a base material.

### BRIEF DESCRIPTION OF THE DRAWINGS

FIG. 1 is a schematic perspective view showing a configuration example of a medical device according to an embodiment of the present invention.

FIG. 2 is a schematic plan view showing a part of an indicator in a resin molding for a medical device of an embodiment of the present invention.

FIG. 3 is a cross-sectional view taken along a line A-A in FIG. 2.

FIG. 4 is a schematic plan view showing a part of an indicator in a resin molding for a medical device of a first modified example of an embodiment of the present invention.

FIG. 5 is a schematic plan view showing a part of an indicator in a resin molding for a medical device of a second modified example of an embodiment of the present invention.

### DETAILED DESCRIPTION OF THE PREFERRED EMBODIMENT

A description will be given below of the medical device and the resin molding for a medical device of embodiments of the present invention with reference to the accompanying drawings. In all the drawings, the same reference numerals are given to the same or corresponding members and a repeated description thereof is omitted here.

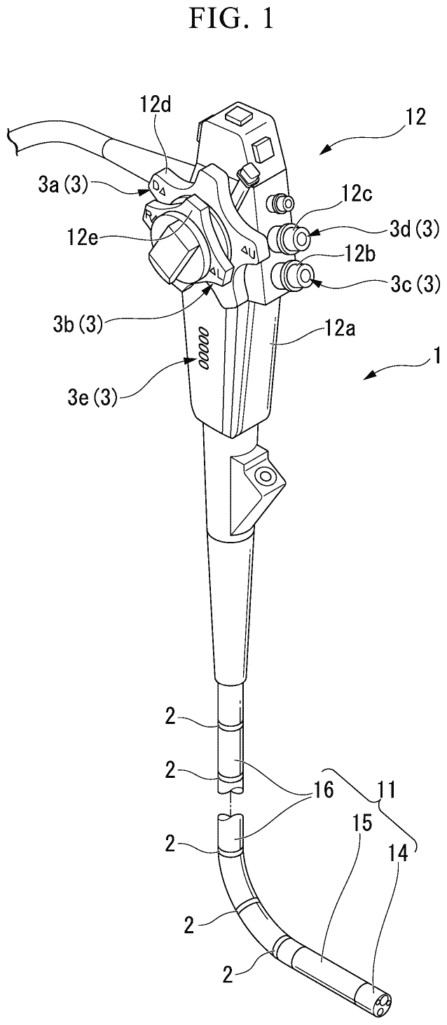

FIG. 1 is a schematic perspective view showing a configuration example of a medical device of an embodiment of the present invention.

As shown in FIG. 1, an endoscope 1 (medical device) of the present embodiment is provided with an insertion portion 11 and an operation portion 12.

The insertion portion 11 is inserted into the body of a patient. The insertion portion 11 is tubular. The insertion portion 11 has flexibility. The insertion portion 11 has a tip portion 14, a bending portion 15, and a flexible tube portion 16 in this order from the tip side in the insertion direction. Although not particularly shown, a treatment tool channel may be provided inside the insertion portion 11 in the longitudinal direction. The treatment tool channel is formed of a tube or lumen. A treatment tool is inserted through the treatment tool channel.

The tip portion 14 is arranged at the furthest tip portion of the endoscope 1. The tip portion 14 has a columnar outer shape. The tip portion 14 is provided with an end effector which functions as a manipulator.

In the present embodiment, the tip portion 14 includes an imaging element and an imaging optical system inside. The imaging element acquires an image of a subject. As the imaging element, for example, a CCD or the like may be used. The imaging optical system is provided with a lens.

At the tip of the tip portion 14, an imaging window and an illumination window are formed. In a case where the insertion portion 11 is provided with a treatment tool channel, an opening of the treatment tool channel is provided at the tip of the tip portion 14.

A bending portion 15 is connected to a base end side of the tip portion 14. The bending portion 15 changes the direction of the tip portion 14. The bending portion 15 is a tubular portion which is able to bend.

The bending portion 15 includes, for example, a plurality of joint rings. The joint rings are annular. The joint rings are connected to adjacent joint rings to be able to rotate. In the bending portion 15, a plurality of angle wires are inserted inside the joint rings.

Furthermore, inside the bending portion 15, for example, members such as electric wiring and a light guide are accommodated. The electric wiring is connected to the imaging element of the tip portion 14. The light guide extends to the illumination window.

The members such as the electric wiring and the light guide are inserted into the flexible tube portion 16 described below. The members such as the electric wiring and the light guide extend to the operation portion 12 described below.

The flexible tube portion 16 is a tubular portion which connects the bending portion 15 and the operation portion 12 described below.

The flexible tube portion 16 includes, for example, a corrugated tube and a sheath resin. The corrugated tube is a member in which a band-shaped member is wound in a spiral shape. The band-shaped member is made of metal or resin. The sheath resin has flexibility. The sheath resin covers the outer peripheral portion of the corrugated tube in a tubular shape.

As the sheath resin, for example, one or more types of resins selected from the group consisting of styrene-based resins, olefin-based resins, vinyl-based chloride resins, polyester-based resins, polyurethane-based resins, and nylon-based resins may be used.

It is possible for the flexible tube portion 16 to bend in an appropriate direction in a state where a substantially circular cross-section is maintained.

Although not particularly shown, at least two systems of angle wires are arranged inside the flexible tube portion 16. The angle wires include at least a first angle wire and a second angle wire. Each angle wire is inserted in a coil sheath. Each angle wire extends from the bending portion 15 to the base end side.

The members such as the electric wiring and the light guide described above are inserted into the inside of the flexible tube portion 16 in the same manner as the bending portion 15.

Indicators 2 are formed on the flexible tube portion 16. The indicators 2 are visible from the outside. The indicators 2 are marks provided for the purpose of allowing an operator to easily grasp the length of the insertion portion 11 inserted into the body of a patient.

The formation positions, shapes, and number of the indicators 2 are not particularly limited. In the present embodiment, as an example, the indicators 2 are annular marks. The indicators 2 are around the outer peripheral portion of the flexible tube 16. The indicators 2 are arranged at equal intervals in the longitudinal direction of the flexible tube portion 16. Although not shown in FIG. 1, numerals, characters, symbols, or the like may be drawn as the indicators 2 in addition to annular marks. Numerals, characters, symbols, or the like may represent the length from the tip of the tip portion 14. The indicators 2 may be drawn as the numerals, characters, symbols, or the like, instead of annular marks.

The operation portion 12 is an apparatus portion with which the operator operates the endoscope 1. Examples of operations performed through the operation portion 12 include an operation of pulling the angle wire for the purpose of changing the bending amount of the bending portion 15. The operation portion 12 has an operation portion main body 12a. Various operation members are provided on the operation portion main body 12a. For example, the various operation members may be operation knobs, operation switches, and the like.

A resin molded article is used as the operation portion main body 12a and at least a part of the operation members. Here, the resin molded article includes a molded article formed of only a resin and a molded article formed of a composite material of a resin and a metal. For example, in the example shown in FIG. 1, as operation members, an air supply and water supply button 12b, a suction button 12c, a first angle knob 12d, a second angle knob 12e, and the like are provided.

The air supply and water supply button 12b is used in an operation for injecting air or water from an air supply and water supply nozzle (not shown). The air supply and water supply nozzle (not shown) is open at the tip portion 14. Air or water is sent from a tank (not shown) through an air supply and water supply pipe (not shown). The air supply and water supply pipe (not shown) is arranged in the insertion portion 11.

The suction button 12c is used in an operation for suctioning liquid around the tip portion 14. For example, the liquid is suctioned through a tubular portion such as a treatment tool channel in the insertion portion 11.

The first angle knob 12d pulls the first angle wire. When the first angle knob 12d is rotated, the bending portion 15 bends in a first direction. For example, the first direction is the up-down direction.

The second angle knob 12e pulls the second angle wire. When the second angle knob 12e is rotated, the bending portion 15 bends in a second direction. For example, the second direction is the left-right direction.

Various indicators 3 are formed on the operation portion 12.

For example, the first angle knob 12d is provided with display marks 3a indicating the operation direction of the bending portion 15. The display marks 3a are examples of the indicators 3.

“DA” and “AU” are drawn on the display marks 3a shown in FIG. 1. The letters “D” and “U” represent downward and upward, respectively. The symbol “A” indicates the rotation direction of the first angle knob 12d in the operation for moving the bending portion 15 downward and upward.

In the same manner, display marks 3b indicating the operation direction of the bending portion 15 are formed on the second angle knob 12e. The display marks 3b are examples of the indicator 3.

“RΔ” and “ΔL” are drawn on the display marks 3b shown in FIG. 1. The letters “R” and “L” represent right and left, respectively.

For example, markers 3c and 3d are formed on the air supply and water supply button 12b and the suction button 12c. The markers 3c and 3d are examples of the indicators 3.

The markers 3c and 3d are display marks provided for the purpose of distinguishing the air supply and water supply button 12b and the suction button 12c. For example, the markers 3c and 3d are colored differently from each other. For example, the markers 3c and 3d may be formed of figures having different shapes.

For example, a logo 3e is formed on the operation portion main body 12a. The logo 3e is an example of the indicator 3. For example, the logo 3e indicates a model name, a model number, a manufacturer name, and the like. The logo 3e may include, for example, symbols, characters, figures, and the like.

The first angle knob 12d, the second angle knob 12e, the air supply/water supply button 12b, the suction button 12c, and the operation portion main body 12a on which the indicators 3 are formed each form the resin molding of the present embodiment in the endoscope 1 along with each of the indicators 3.

A description will be given below focusing on the configuration common to each resin molding.

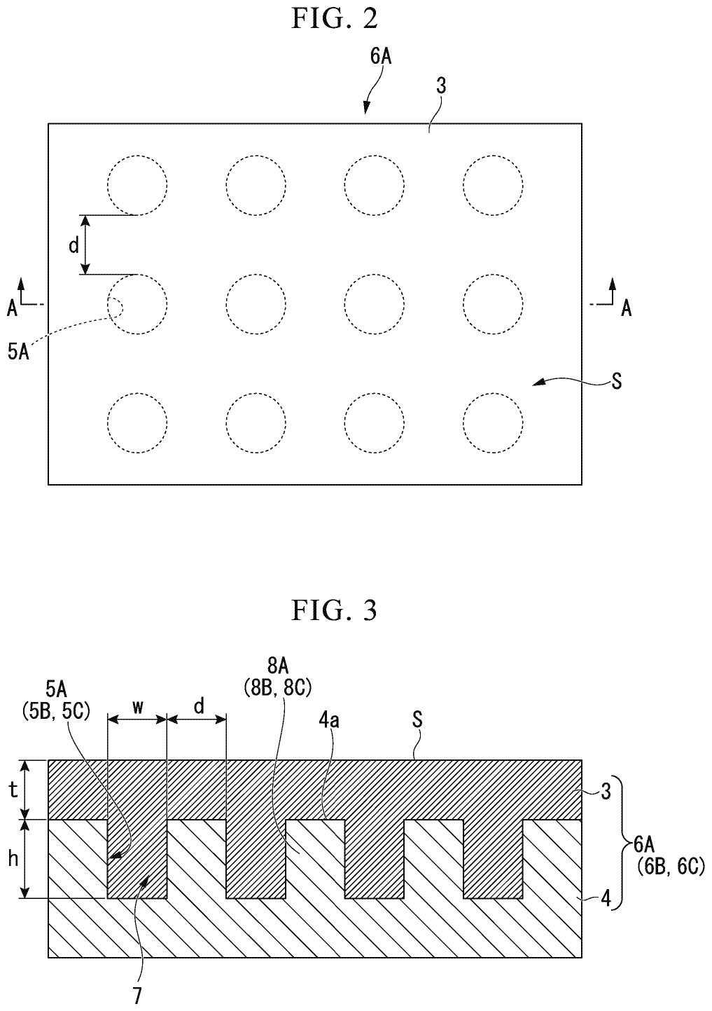

FIG. 2 is a schematic plan view showing a part of an indicator in a resin molding for a medical device of an embodiment of the present invention. FIG. 3 is a cross-sectional view taken along line A-A in FIG. 2.

As shown in FIGS. 2 and 3, a resin molding 6A of the present embodiment is provided with the indicator 3 and a base material 4 (refer to FIG. 3). In FIGS. 2 and 3, the indicator 3 and a part of a surface layer portion of the base material 4 are shown in an enlarged manner.

As shown in FIG. 3, the indicator 3 has a flat surface S. The indicator 3 covers the base material 4.

The indicator 3 is formed of a cured product of a UV-curable ink. However, the UV-curable ink may include a solid component when not cured. The solid component becomes a part of the cured product after curing of the UV-curable ink.

The UV-curable ink includes at least a base resin and a photopolymerization initiator.

The UV-curable ink includes an appropriate coloring material as necessary. The coloring material may be a pigment or a dye. Other components in the UV-curable ink may include, for example, a polymerization inhibitor, a reactive diluent, a fluorescent brightener, and the like.

The material of the UV-curable ink is not particularly limited as long as the material has durability with respect to sterilization processes after curing. For example, examples of suitable UV-curable ink materials for the indicator 3 include acrylic-based inks, epoxy-based inks, urethane-based inks, and the like.

The indicator 3 is manufactured by printing the UV-curable ink on the surface of the base material 4 and then UV-curing the ink. The UV-curable ink is cured by irradiation with UV light. It is not necessary to heat the UV-curable ink by a heating furnace or the like during curing. As a result, it is possible to cure the UV-curable ink with simple equipment. The time required for complete curing of the UV-curable ink is shorter than for thermosetting ink. As a result, it is possible to rapidly cure the UV-curable ink.

The method of printing the UV-curable ink is not particularly limited. Examples of the method of printing the UV-curable ink include ink jet printing, coating printing with a dispenser, and the like.

Medical devices are often produced in various types and small quantities. A printing plate is not necessary for the ink jet printing, coating printing with a dispenser, and the like. Ink jet printing, coating printing with a dispenser, and the like are particularly suitable for production involving various types and small quantities.

The thickness of the indicator 3 is not particularly limited as long as the mechanical strength of the indicator 3 is secured. For example, the thickness t+h of the indicator 3 may be 70 μm or more and 300 μm or less. Here, t is the thickness of the indicator 3 outside the surface 4a. h is the thickness in the inside portion of the base material 4 described below.

The area of the indicator 3 is not particularly limited. However, if the width of the indicator 3 in plan view is excessively narrow, the number of the circular holes 5A covered by the indicator 3 is excessively small. For example, the width of the indicator 3 is more preferably 0.3 mm or more.

The base material 4 is a resin portion on the surface of the first angle knob 12d, the second angle knob 12e, the air supply and water supply button 12b, the suction button 12c, and the operation portion main body 12a.

For example, the base material 4 is manufactured using resin molding. The method for molding the base material 4 is not particularly limited. Examples of the method for molding the base material 4 include injection molding, extrusion molding, blow molding, press molding, and the like.

The material of the base material 4 is not particularly limited as long as the material is a resin material having adhesion to the UV-curable ink which is the raw material of the indicator 3.

For example, as the base material 4, a resin material including one or more resins selected from the group consisting of polytetrafluoroethylene (PTFE), tetrafluoroethylene hexafluoropropylene resin (FEP), polyethylene, polyolefin, modified polyphenylene oxide, polyamide, vinyl chloride, latex, natural rubber, polysulfone, polyphenylsulfone, polyetherimide, polyacetal (POM), polyetheretherketone (PEEK), polycarbonate, and acrylonitrile-butadiene-styrene (ABS), as a component, may be used.

For example, the base material 4 may be a film molding formed of one or more resins selected from the group consisting of an epoxy-based resin, a polyurethane-based resin, an acrylic-based resin, a polyester-based resin, and a fluorine-based resin. As the acrylic-based resin, for example, an acrylic-based paint, an acrylic-based silicone paint, or an acrylic-based urethane paint may be used.

The method of molding the film molding is not particularly limited. For example, the surface of a resin or metal substrate may be coated with a raw material including a resin which becomes a film molding after curing. In such a case, the film product is molded into a shape along the surface shape of the substrate. The shape of the substrate is not particularly limited.

For example, as a coating method for forming a film molding, electrodeposition coating, electrostatic coating, powder coating, ultraviolet curing coating, baking coating, or the like may be used.

An uneven structure is formed on the surface of the base material 4 on which the indicator 3 is formed (at the interface with the indicator 3).

The uneven structure includes at least a plurality of recesses having a width of 10 μm or more and 60 μm or less.

More preferably, the uneven structure is formed in a range overlapping with the indicator 3. However, the uneven structure may be formed in a range wider than the indicator 3.

For example, the recess in the uneven structure may be formed by a hole or a groove depressed from the surface 4a. In a case where the recess is a hole, the width of the recess is defined according to the smallest hole diameter. In a case where the recess is a groove, the width of the recess is defined by the groove width in a cross-section orthogonal to the extending direction of the groove.

When the recess described above is formed on the surface 4a, the material of the indicator 3 enters the recess. As a result, the contact area between the indicator 3 and the base material 4 increases. As a result of the increase in the contact area between the indicator 3 and the base material 4, the adhesion strength of the indicator 3 to the base material 4 is improved. Furthermore, the indicator 3 which entered the recess is interposed between the inner peripheral surfaces of the recess. As a result, the indicator 3 which entered the recess is firmly joined to the recess. As a result of the indicators 3 which entered the recess being firmly joined to the recess, the adhesion strength of the indicators 3 to the base material 4 is improved.

When the width of the recess is less than 10 μm, it is difficult for UV-curable ink to enter the recess. As a result, there is a concern that the adhesion strength of the indicator 3 may be reduced.

When the width of the recess exceeds 60 μm, the number of recesses in the region for forming the indicator 3 may be excessively small in relative terms. In such a case, as a result of a reduction in the contact area between the indicator 3 and the base material 4, there is a concern that the adhesion strength of the indicator 3 may be reduced.

The depth of the recess is more preferably 5 μm or more and 60 μm or less.

When the depth of the recess is less than 5 μm, as a result of the amount of the UV-curable ink embedded in the recess being excessively small, there is a concern that the adhesion strength of the indicator 3 may be reduced.

When the depth of the recess exceeds 60 μm, there is a concern that it may be difficult for the UV-curable ink to easily enter the bottom of the recess. In such a case, as a result of a reduction in the contact area between the indicator 3 and the base material 4, there is a concern that the adhesion strength of the indicator 3 may be reduced.

The interval between the recesses adjacent to each other is more preferably 1 μm or more and 30 μm or less.

When the interval between the recesses is less than 1 μm, the thickness of the resin wall interposed between the recesses becomes excessively thin. As a result, there is a concern that the strength of the resin wall may be excessively low. In such a case, there is a concern that the indicator 3 may be easily peeled off due to breakage of the resin wall.

When the interval between the recesses exceeds 30 μm, there is a concern that the number of recesses in the region for forming the indicator 3 may be excessively small in relative terms. As a result, there is a concern that the contact area between the indicator 3 and the base material 4 may be reduced such that the adhesion strength of the indicator 3 is reduced.

The plurality of recesses may be arranged periodically or may be arranged aperiodically.

In a case where a plurality of recesses are periodically arranged, the plurality of recesses may be arranged with a single spatial period, or may be arranged in a periodic arrangement in which two or more spatial periods are mixed. For example, in the case of an arrangement including a plurality of spatial periods, the spatial periods may be different depending on the direction.

The shape of the recess is not particularly limited.

For example, in the examples shown in FIGS. 2 and 3, the recess is a circular hole 5A (recess) having a diameter w (width) and a depth h. As shown in FIG. 2, the circular holes 5A are arranged in a square lattice. The circular holes 5A are arranged at equal pitches in two directions orthogonal to each other. The interval of each circular hole 5A in each arrangement direction is d, and each arrangement pitch is w+d.

In such a case, the interval between the circular holes 5A adjacent to each other is d in the arrangement direction. The interval between the circular holes 5A adjacent to each other is approximately 1.4 times (√2 times) d in the diagonal direction of the lattice.

The diameter w of the circular hole 5A is set to 10 μm or more and 60 μm or less.

In FIG. 3, the bottom surface of the circular hole 5A is illustrated as a plane parallel to the surface 4a. However, the bottom surface of the circular hole 5A is not limited to a plane. For example, the bottom surface of the circular hole 5A may be a curved surface, a mortar surface, a fine uneven surface, or the like. The depth of the circular hole 5A is defined by the distance between the surface 4a and the deepest part of the bottom surface.

The circular hole 5A is depressed from the surface 4a. A wall portion 8A is formed between the circular holes 5A which are adjacent to each other. The wall portion 8A is a projection higher than the bottom surface of the circular hole 5A. As a result, an uneven structure in which recesses and projections are mixed is formed in the surface layer portion of the base material 4.

For example, the resin molding 6A is manufactured as follows.

First, the base material 4 is manufactured.

The base material 4 may be manufactured by resin molding using a mold. The base material 4 may be manufactured by molding a film molding on the surface of an appropriately shaped substrate.

The circular hole 5A of the base material 4 is formed on the surface 4a during the molding or after the molding of the base material 4.

For example, in a case where the circular hole 5A is formed during the molding of the base material 4, a mold for transferring the shape of the circular hole 5A may be used.

For example, in a case where the circular hole 5A is formed after the molding of the base material 4, the circular hole 5A may be formed by laser processing.

UV-curable ink is printed on the surface 4a where the circular hole 5A is formed.

For example, the UV-curable ink is printed in the shape of the indicator 3 by an ink jet apparatus. The UV-curable ink enters the inside of the circular hole 5A. When the circular hole 5A is filled with the UV-curable ink, the remaining UV-curable ink forms a layered portion on the circular hole 5A and the surface 4a.

The printing thickness of the UV-curable ink is a thickness which is able to form a layer with a thickness necessary for the indicator 3 when curing the UV-curable ink.

The printed UV-curable ink is irradiated with UV light (ultraviolet light). The UV-curable ink is cured when irradiated with UV light. In this manner, the indicator 3 is formed.

An appropriate coating layer may be provided on the indicator 3 and the base material 4 not covered with the indicator 3, as necessary. Through the above, the resin molding 6A is manufactured.

In the resin molding 6A, the indicator 3 is cured in a state of adhering to the surface 4a and the inner peripheral surface of the circular hole 5A. The UV-curable ink cured inside the circular hole 5A has a projection 7 fitted into the circular hole 5A. The projection 7 increases the contact area between the indicator 3 and the base material 4 in comparison with a case where the circular hole 5A is not present. As a result, the adhesion between the indicator 3 and the base material 4 is improved.

Furthermore, the projection 7 is in a state of being fitted into the circular hole 5A. The projection 7 obtains an anchor effect. For example, in a case where an external force acts to deform the indicator 3 and the base material 4, the projection 7 increases the resistance to pulling out from the circular hole 5A. As a result, the durability of the indicator 3 is improved.

Furthermore, forming the projection 7 gives the interface between the indicator 3 and the base material 4 an uneven surface. For example, in a case where the indicator 3 and the base material 4 are bonded to each other on a plane, when an agent or a sterilizing gas for a sterilization process enters a part of the interface, cracks expand along a plane and the penetration range is easily expanded. However, in the present embodiment, the interface is an uneven surface. As a result, even if a crack develops from the part of the penetration point, the projection 7 prevents the crack from developing. Accordingly, in comparison with a case where the indicator 3 and the base material 4 are bonded to each other on a plane, the present embodiment is more likely to suppress the spreading of the penetration range of the agent and sterilizing gas of the sterilization process. As a result, peeling of the indicator 3 due to repetition of the sterilization process is more easily suppressed.

As described above, it is possible to manufacture the resin molding 6A of the present embodiment quickly and easily, and the adhesion between the indicator and the base material is improved.

### First Modified Example

Next, a description will be given of a first modified example of the present embodiment.

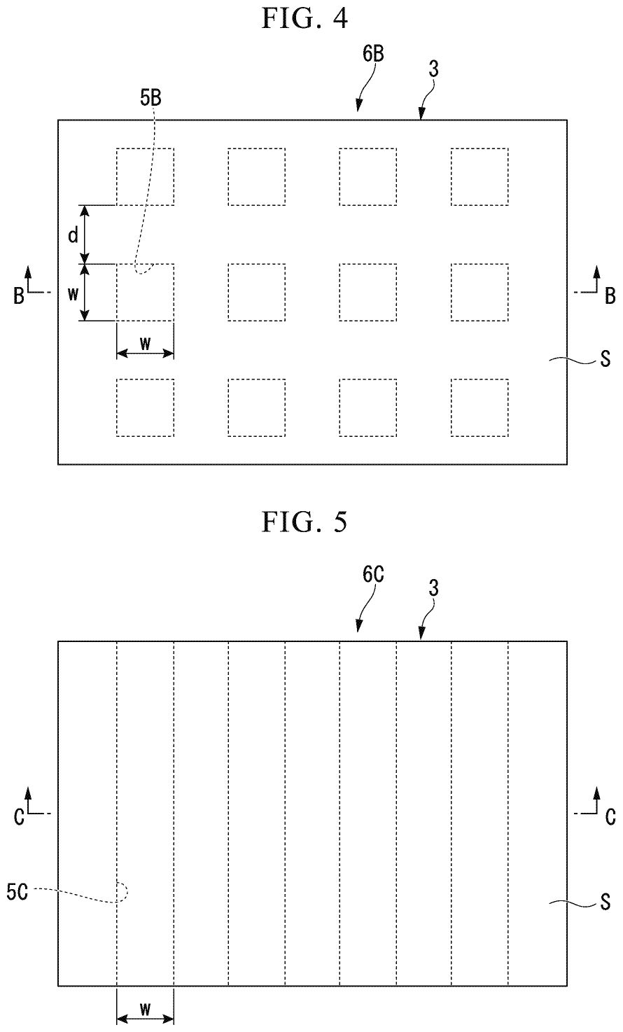

FIG. 4 is a schematic plan view showing a part of the indicator in the resin molding for a medical device of a first modified example of the embodiment of the present invention.

As shown in FIG. 3, a resin molding 6B of the present modified example is provided with square holes 5B (recesses) and wall portions 8B, instead of the circular holes 5A and the wall portions 8A of the resin molding 6A of the embodiment described above. However, the cross-section of FIG. 3 in the present modified example corresponds to the cross-section along the line B-B in FIG. 4. It is possible to use the resin molding 6B for the operation portion 12 and the like of the endoscope 1 of the embodiment described above, in the same manner as the resin molding 6A.

A description will be given below mainly of the points different from the embodiment described above.

As shown in FIGS. 3 and 4, the square hole 5B is a recess having a length (width) w on one side of an opening and a depth h. The square hole 5B is depressed from the surface 4a. The square holes 5B are arranged in a square lattice. The square holes 5B are arranged at equal pitches in two directions orthogonal to each other. Each interval in each arrangement direction of each of the square holes 5B is d, and each arrangement pitch is w+d.

The interval between the square holes 5B which are adjacent to each other is approximately 1.4 times (√2 times) d in the diagonal direction of the lattice.

In the same manner as the circular hole 5A, the bottom surface of the square hole 5B is not limited to a plane. For example, the bottom surface of the square hole 5B may be a curved surface, a mortar surface, a fine uneven surface, or the like. The depth of the square hole 5B is defined by the distance between the surface 4a and the deepest part of the bottom surface.

A wall portion 8B is formed between the adjacent square holes 5B. The wall portion 8B is a projection higher than the bottom surface of the square hole 5B. In the surface layer portion of the base material 4, an uneven structure in which recesses and projections are mixed is formed.

Thus, the resin molding 6B of the present modified example is configured in the same manner as the above embodiment, except that the shape of the recess in plan view is changed to a square.

The resin molding 6B is manufactured in the same manner as the resin molding 6A of the embodiment described above, except that the shape of the recess in plan view is different.

The resin molding 6B of the present modified example is able to be manufactured quickly and easily in the same manner as the resin molding 6A of the embodiment described above, and the adhesion between the indicator and the base material is improved.

### Second Modified Example

Next, a description will be given of a second modified example of the present embodiment.

FIG. 5 is a schematic plan view showing, in an enlarged manner, a part of an indicator in a resin molding for a medical device of a second modified example of the embodiment of the present invention.

As shown in FIG. 3, a resin molding 6C of the present modified example is provided with a linear groove 5C (recess) and a wall portion 8C, instead of the circular hole 5A and the wall portion 8A of the resin molding 6A of the embodiment described above. However, the cross-section of FIG. 3 in the present modified example corresponds to the cross-section along the line C-C in FIG. 5. It is possible to use the resin molding 6C for the operation portion 12 and the like of the endoscope 1 of the above embodiment, in the same manner as the resin molding 6A.

A description will be given below mainly of the points different from the embodiment described above.

As shown in FIGS. 3 and 5, the linear groove 5C is a recess having a groove width (width) w and a depth h. The linear groove 5C is depressed from the surface 4a. The linear grooves 5C are arranged in parallel with each other. The linear grooves 5C are arranged at an equal pitch in the arrangement direction orthogonal to the extending direction. The interval in the arrangement direction of each linear groove 5C is d, and the arrangement pitch is w+d.

In the same manner as the circular hole 5A, the bottom surface of the linear groove 5C is not limited to a plane. For example, the bottom surface of the linear groove 5C may be a curved surface, a mortar surface, a fine uneven surface, or the like. The depth of the linear groove 5C is defined by the distance between the surface 4a and the deepest portion of the bottom surface.

A wall portion 8C is formed between the adjacent linear grooves 5C. The wall portion 8C is a projection higher than the bottom surfaces of the linear grooves 5C. In the surface layer portion of the base material 4, an uneven structure in which recesses and projections are mixed is formed.

Thus, the resin molding 6C of the present modified example is configured in the same manner as the above embodiment, except that the shape of the recess in plan view is changed to a linear shape extending in one direction.

The resin molding 6C is manufactured in the same manner as the resin molding 6A of the embodiment described above except that the shape of the recess in plan view is different.

The resin molding 6C of the present modified example is able to be manufactured quickly and easily in the same manner as the resin molding 6A of the embodiment described above, and the adhesion between the indicator and the base material is improved.

In the description of the embodiment described above, an example of a case in which the medical device was the endoscope 1 was described. However, medical devices in which it is possible to use the resin molding for a medical device of the present invention are not limited to endoscopes. The resin molding for a medical device of the present invention may be used for a medical device such as a treatment tool, a syringe, and a cleaning device.

In the description of the embodiment and each of the modified examples described above, an example of a case in which one type of hole or one type of groove was used as the recess in the base material of the resin molding was described.

However, a mixture of a plurality of types of recess holes may be used. A mixture of a plurality of types of recess grooves may be used. Furthermore, as the recesses, a mixture of holes and grooves may be used.

In the description of the embodiment and each of the modified examples described above, examples of cases in which the circular holes 5A, the square holes 5B, and the linear grooves 5C with a linear shape were used as recesses in the base material of the resin molding were described. However, the shape of the recess is not limited thereto.

For example, in a case where the recess is a hole, for the shape in plan view, an ellipse, an oval (a shape like a track in an athletic stadium), a polygon other than a square, a concave polygon, or the like may be used.

For example, in a case where the recess is a groove, the shape in plan view may be a curved shape, a zigzag shape, a sawtooth shape, or the like.

In the description of the embodiment and the first modified example described above, an example of a case in which the recesses are arranged in a square lattice shape in plan view was described. However, the arrangement of the recesses is not limited to a square lattice. For example, the recesses may be arranged in a rectangular lattice other than a square lattice, an oblique lattice, concentric circles, a radial shape, or the like.

In the description of the embodiment and each of the modified examples described above, an example of a case in which the resin molding for a medical device was used for the operation portion 12 was described. However, the resin molding for a medical device may be used for any device portion of the endoscope 1. For example, a resin molding for a medical device may be used for the insertion portion 11. For example, a sheath resin or a surface coat layer of the insertion portion 11 may be used for the base material. In such a case, it is sufficient if an uneven structure is provided in the forming range of the indicator 2. The indicator 2 is formed of a UV-curable ink. Due to this, the adhesion between the indicator 2 and the sheath resin or the surface coat layer of the insertion portion 11 is improved.

In the second modified example, an example of a case in which linear grooves were provided in parallel with each other was described. However, the linear grooves may be provided so as to be orthogonal to each other. For example, the linear grooves may be arranged in a rectangular lattice, an oblique lattice, or the like. In such a case, the portions surrounded by the linear groove in the base material are projections higher than the groove bottom of the linear groove. The shape of the projections is columnar.

In the description of the embodiment and each of the modified examples described above, examples of the method of processing the uneven structure include molding and laser processing. However, the processing method of the uneven structure is not limited to these processing methods.

For example, the uneven structure may be formed by pressing processing, blasting processing, etching processing, or the like.

### EXAMPLES

Next, a description will be given of Examples of the resin molding for a medical device of the embodiment described above and each modified example together with Comparative Examples.

The following Table 1 shows the base material configurations and evaluation results of Examples 1-1 to 1-27. Examples 1-1 to 1-27 are all Examples of the resin molding 6A of the embodiment.

TABLE 1

| | | | Recess | Recess | Recess | Cross- | Alcohol |
|---|---|---|---|---|---|---|---|
| | Base | Recess | depth | width | interval | cut | wiping |
| | material | shape | (μm) | (μm) | (μm) | test | test |
| Example 1-1 | Polysulfone | Circular hole | 5 | 10 | 1 | 2 | Good |
| Example 1-2 | Polysulfone | Circular hole | 5 | 10 | 10 | 1 | Good |
| Example 1-3 | Polysulfone | Circular hole | 5 | 10 | 20 | 0 | Good |
| Example 1-4 | Polysulfone | Circular hole | 5 | 30 | 1 | 0 | Good |
| Example 1-5 | Polysulfone | Circular hole | 5 | 30 | 10 | 0 | Good |
| Example 1-6 | Polysulfone | Circular hole | 5 | 30 | 20 | 0 | Good |
| Example 1-7 | Polysulfone | Circular hole | 5 | 60 | 1 | 1 | Good |
| Example 1-8 | Polysulfone | Circular hole | 5 | 60 | 10 | 1 | Good |
| Example 1-9 | Polysulfone | Circular hole | 5 | 60 | 20 | 1 | Good |
| Example 1-10 | Polysulfone | Circular hole | 30 | 10 | 1 | 0 | Good |
| Example 1-11 | Polysulfone | Circular hole | 30 | 10 | 10 | 0 | Good |
| Example 1-12 | Polysulfone | Circular hole | 30 | 10 | 20 | 0 | Good |
| Example 1-13 | Polysulfone | Circular hole | 30 | 30 | 1 | 0 | Good |
| Example 1-14 | Polysulfone | Circular hole | 30 | 30 | 10 | 0 | Good |
| Example 1-15 | Polysulfone | Circular hole | 30 | 30 | 20 | 0 | Good |
| Example 1-16 | Polysulfone | Circular hole | 30 | 60 | 1 | 0 | Good |
| Example 1-17 | Polysulfone | Circular hole | 30 | 60 | 10 | 0 | Good |
| Example 1-18 | Polysulfone | Circular hole | 30 | 60 | 20 | 1 | Good |
| Example 1-19 | Polysulfone | Circular hole | 60 | 10 | 1 | 0 | Good |
| Example 1-20 | Polysulfone | Circular hole | 60 | 10 | 10 | 0 | Good |
| Example 1-21 | Polysulfone | Circular hole | 60 | 10 | 20 | 0 | Good |
| Example 1-22 | Polysulfone | Circular hole | 60 | 30 | 1 | 0 | Good |
| Example 1-23 | Polysulfone | Circular hole | 60 | 30 | 10 | 0 | Good |
| Example 1-24 | Polysulfone | Circular hole | 60 | 30 | 20 | 0 | Good |
| Example 1-25 | Polysulfone | Circular hole | 60 | 60 | 1 | 2 | Good |
| Example 1-26 | Polysulfone | Circular hole | 60 | 60 | 10 | 2 | Good |
| Example 1-27 | Polysulfone | Circular hole | 60 | 60 | 20 | 2 | Good |

### Example 1-1

As shown in Table 1, as the material of the base material 4 in a test sample of Example 1-1 (the symbol is omitted in Table 1 and in the other tables), polysulfone was used. As the base material 4 of the test sample, a flat surface portion member provided with a flat surface portion having a surface having a size of 20 mm×20 mm was used. The thickness of the base material 4 was 5 mm.

The base material 4 was manufactured by injection molding.

A large number of circular holes 5A were formed on the surface 4a of the base material 4 in a range of 19 mm×19 mm.

The diameter w (recess width in Table 1) of each circular hole 5A was 10 μm. The depth h (recess depth in Table 1) of each circular hole 5A was 5 μm. The interval d between the arrangements of each of the circular holes 5A (the interval between recesses in Table 1) was 1 μm.

Laser processing was used as a processing method for the circular holes 5A. Specifically, a laser marker MD-S9900A (trade name; manufactured by KEYENCE CORPORATION) was used.

The indicator 3 was formed on the surface 4a of the base material 4 on which the circular holes 5A were formed.

The shape of the indicator 3 in plan view was “RΔ”. The line width of “R” in the indicator 3 was approximately 1.5 mm. The inside of “Δ” in the indicator 3 was filled. The region for forming the indicator 3 had a size of 8 mm×8 mm within a range of 19 mm×19 mm in which the circular holes 5A were distributed.

The indicator 3 was printed on the surface 4a by an ink jet apparatus. The thickness t of the indicator 3 on the surface 4a was 150 μm.

### Examples 1-2 and 1-3

The resin moldings for a medical device 6A of Examples 1-2 and 1-3 were manufactured in the same manner as in Example 1-1 except that the interval d of the arrangement of the circular holes 5A in Example 1-1 was changed to 10 μm and 20 μm, respectively.

### Examples 1-4 to 1-9

The resin moldings for a medical device 6A of Examples 1-4 to 1-6 were manufactured in the same manner as in Examples 1-1 to 1-3 except that the recess widths in Examples 1-1 to 1-3 were each changed to 30 μm.

The resin moldings for a medical device 6A of Examples 1-7 to 1-9 were manufactured in the same manner as in Examples 1-1 to 1-3 except that the recess widths in Examples 1-1 to 1-3 were each changed to 60 μm.

### Examples 1-10 to 1-18

The resin moldings for a medical device 6A of Examples 1-10 to 1-18 were manufactured in the same manner as in Examples 1-1 to 1-9, except that the recess depths in Examples 1-1 to 1-9 were each changed to 30 μm.

### Examples 1-19 to 1-27

The resin moldings for a medical device 6A of Examples 1-19 to 1-27 were manufactured in the same manner as in Examples 1-1 to 1-9, except that the recess depths in Examples 1-1 to 1-9 were each changed to 60 μm.

### Examples 2-1 to 2-27

The following Table 2 shows the base material configurations and evaluation results of Examples 1-1 to 1-27. Examples 2-1 to 2-27 are all Examples of the resin molding 6B of the first modified example.

TABLE 2

| | | | Recess | Recess | Recess | Cross- | Alcohol |
|---|---|---|---|---|---|---|---|
| | Base | Recess | depth | width | interval | cut | wiping |
| | material | shape | (μm) | (μm) | (μm) | test | test |
| Example 2-1 | Polysulfone | Square hole | 5 | 10 | 1 | 2 | Good |
| Example 2-2 | Polysulfone | Square hole | 5 | 10 | 10 | 1 | Good |
| Example 2-3 | Polysulfone | Square hole | 5 | 10 | 20 | 0 | Good |
| Example 2-4 | Polysulfone | Square hole | 5 | 30 | 1 | 1 | Good |
| Example 2-5 | Polysulfone | Square hole | 5 | 30 | 10 | 0 | Good |
| Example 2-6 | Polysulfone | Square hole | 5 | 30 | 20 | 0 | Good |
| Example 2-7 | Polysulfone | Square hole | 5 | 60 | 1 | 1 | Good |
| Example 2-8 | Polysulfone | Square hole | 5 | 60 | 10 | 1 | Good |
| Example 2-9 | Polysulfone | Square hole | 5 | 60 | 20 | 1 | Good |
| Example 2-10 | Polysulfone | Square hole | 30 | 10 | 1 | 0 | Good |
| Example 2-11 | Polysulfone | Square hole | 30 | 10 | 10 | 0 | Good |
| Example 2-12 | Polysulfone | Square hole | 30 | 10 | 20 | 0 | Good |
| Example 2-13 | Polysulfone | Square hole | 30 | 30 | 1 | 0 | Good |
| Example 2-14 | Polysulfone | Square hole | 30 | 30 | 10 | 0 | Good |
| Example 2-15 | Polysulfone | Square hole | 30 | 30 | 20 | 0 | Good |
| Example 2-16 | Polysulfone | Square hole | 30 | 60 | 1 | 0 | Good |
| Example 2-17 | Polysulfone | Square hole | 30 | 60 | 10 | 0 | Good |
| Example 2-18 | Polysulfone | Square hole | 30 | 60 | 20 | 1 | Good |
| Example 2-19 | Polysulfone | Square hole | 60 | 10 | 1 | 0 | Good |
| Example 2-20 | Polysulfone | Square hole | 60 | 10 | 10 | 0 | Good |
| Example 2-21 | Polysulfone | Square hole | 60 | 10 | 20 | 0 | Good |
| Example 2-22 | Polysulfone | Square hole | 60 | 30 | 1 | 0 | Good |
| Example 2-23 | Polysulfone | Square hole | 60 | 30 | 10 | 0 | Good |
| Example 2-24 | Polysulfone | Square hole | 60 | 30 | 20 | 0 | Good |
| Example 2-25 | Polysulfone | Square hole | 60 | 60 | 1 | 2 | Good |
| Example 2-26 | Polysulfone | Square hole | 60 | 60 | 10 | 2 | Good |
| Example 2-27 | Polysulfone | Square hole | 60 | 60 | 20 | 2 | Good |

As shown in Table 2, the test samples of Examples 2-1 to 2-27 were manufactured in the same manner as in Examples 1-1 to 1-27 except that the square holes 5B were provided instead of the circular hole 5A of the base material 4. In Table 2, the depth of the recess of the square hole 5B is the depth h in the square hole 5B. The width of the recess of the square hole 5B is the length w of one side of the opening in the square hole 5B. The interval between the recesses of the square holes 5B is the interval d of the arrangement of the square holes 5B.

The recess depths, recess widths, and recess intervals in Examples 2-1 to 2-27 were the same as the recess depths, recess widths, and recess intervals in Examples 1-1 to 1-27, respectively.

### Examples 3-1 to 3-27

The following Table 3 shows the base material configurations and evaluation results of Examples 3-1 to 3-27. Examples 3-1 to 3-27 are all Examples of the resin molding 6C of the second modified example.

TABLE 3

| | | | Recess | Recess | Recess | Cross- | Alcohol |
|---|---|---|---|---|---|---|---|
| | Base | Recess | depth | width | interval | cut | wiping |
| | material | shape | (μm) | (μm) | (μm) | test | test |
| Example 3-1 | Polysulfone | Linear groove | 5 | 10 | 1 | 2 | Good |
| Example 3-2 | Polysulfone | Linear groove | 5 | 10 | 10 | 1 | Good |
| Example 3-3 | Polysulfone | Linear groove | 5 | 10 | 20 | 1 | Good |
| Example 3-4 | Polysulfone | Linear groove | 5 | 30 | 1 | 0 | Good |
| Example 3-5 | Polysulfone | Linear groove | 5 | 30 | 10 | 0 | Good |
| Example 3-6 | Polysulfone | Linear groove | 5 | 30 | 20 | 0 | Good |
| Example 3-7 | Polysulfone | Linear groove | 5 | 60 | 1 | 1 | Good |
| Example 3-8 | Polysulfone | Linear groove | 5 | 60 | 10 | 1 | Good |
| Example 3-9 | Polysulfone | Linear groove | 5 | 60 | 20 | 2 | Good |
| Example 3-10 | Polysulfone | Linear groove | 30 | 10 | 1 | 0 | Good |
| Example 3-11 | Polysulfone | Linear groove | 30 | 10 | 10 | 0 | Good |
| Example 3-12 | Polysulfone | Linear groove | 30 | 10 | 20 | 0 | Good |
| Example 3-13 | Polysulfone | Linear groove | 30 | 30 | 1 | 0 | Good |
| Example 3-14 | Polysulfone | Linear groove | 30 | 30 | 10 | 0 | Good |
| Example 3-15 | Polysulfone | Linear groove | 30 | 30 | 20 | 1 | Good |
| Example 3-16 | Polysulfone | Linear groove | 30 | 60 | 1 | 1 | Good |
| Example 3-17 | Polysulfone | Linear groove | 30 | 60 | 10 | 1 | Good |
| Example 3-18 | Polysulfone | Linear groove | 30 | 60 | 20 | 2 | Good |
| Example 3-19 | Polysulfone | Linear groove | 30 | 10 | 1 | 1 | Good |
| Example 3-20 | Polysulfone | Linear groove | 60 | 10 | 10 | 1 | Good |
| Example 3-21 | Polysulfone | Linear groove | 60 | 10 | 20 | 1 | Good |
| Example 3-22 | Polysulfone | Linear groove | 60 | 30 | 1 | 1 | Good |
| Example 3-23 | Polysulfone | Linear groove | 60 | 30 | 10 | 1 | Good |
| Example 3-24 | Polysulfone | Linear groove | 60 | 30 | 20 | 1 | Good |
| Example 3-25 | Polysulfone | Linear groove | 60 | 60 | 1 | 2 | Good |
| Example 3-26 | Polysulfone | Linear groove | 60 | 60 | 10 | 2 | Good |
| Example 3-27 | Polysulfone | Linear groove | 60 | 60 | 20 | 2 | Good |

As shown in Table 3, the test samples of Examples 3-1 to 3-27 were manufactured in the same manner as in Examples 1-1 to 1-27, except that the linear grooves 5C were provided instead of the circular holes 5A of the base material 4. In Table 3, the recess depth of the linear groove 5C is the depth h in the linear groove 5C. The recess width of the linear groove 5C is the groove width w of the linear groove 5C. The interval between the recesses of the linear grooves 5C is the interval d of the arrangement of the linear grooves 5C.

The recess depths, recess widths, and recess intervals in Examples 3-1 to 3-27 were the same as the recess depths, recess widths, and recess intervals in Examples 1-1 to 1-27, respectively.

### Examples 4-1 to 4-27

The following Table 4 shows the base material configurations and evaluation results of Examples 4-1 to 4-27. Examples 4-1 to 4-27 are all Examples of the resin molding 6A of the embodiment.

TABLE 4

| | | | Recess | Recess | Recess | Cross- | Alcohol |
|---|---|---|---|---|---|---|---|
| | Base | Recess | depth | width | interval | cut | wiping |
| | material | shape | (μm) | (μm) | (μm) | test | test |
| Example 4-1 | Acrylic-based coating film | Circular hole | 5 | 10 | 1 | 1 | Good |
| Example 4-2 | Acrylic-based coating film | Circular hole | 5 | 10 | 10 | 1 | Good |
| Example 4-3 | Acrylic-based coating film | Circular hole | 5 | 10 | 20 | 1 | Good |
| Example 4-4 | Acrylic-based coating film | Circular hole | 5 | 30 | 1 | 0 | Good |
| Example 4-5 | Acrylic-based coating film | Circular hole | 5 | 30 | 10 | 0 | Good |
| Example 4-6 | Acrylic-based coating film | Circular hole | 5 | 30 | 20 | 0 | Good |
| Example 4-7 | Acrylic-based coating film | Circular hole | 5 | 60 | 1 | 1 | Good |
| Example 4-8 | Acrylic-based coating film | Circular hole | 5 | 60 | 10 | 1 | Good |
| Example 4-9 | Acrylic-based coating film | Circular hole | 5 | 60 | 20 | 1 | Good |
| Example 4-10 | Acrylic-based coating film | Circular hole | 30 | 10 | 1 | 1 | Good |
| Example 4-11 | Acrylic-based coating film | Circular hole | 30 | 10 | 10 | 0 | Good |
| Example 4-12 | Acrylic-based coating film | Circular hole | 30 | 10 | 20 | 0 | Good |
| Example 4-13 | Acrylic-based coating film | Circular hole | 30 | 30 | 1 | 0 | Good |
| Example 4-14 | Acrylic-based coating film | Circular hole | 30 | 30 | 10 | 0 | Good |
| Example 4-15 | Acrylic-based coating film | Circular hole | 30 | 30 | 20 | 1 | Good |
| Example 4-16 | Acrylic-based coating film | Circular hole | 30 | 60 | 1 | 1 | Good |
| Example 4-17 | Acrylic-based coating film | Circular hole | 30 | 60 | 10 | 1 | Good |
| Example 4-18 | Acrylic-based coating film | Circular hole | 30 | 60 | 20 | 1 | Good |
| Example 4-19 | Acrylic-based coating film | Circular hole | 60 | 10 | 1 | 1 | Good |
| Example 4-20 | Acrylic-based coating film | Circular hole | 60 | 10 | 10 | 0 | Good |
| Example 4-21 | Acrylic-based coating film | Circular hole | 60 | 10 | 20 | 0 | Good |
| Example 4-22 | Acrylic-based coating film | Circular hole | 60 | 30 | 1 | 1 | Good |
| Example 4-23 | Acrylic-based coating film | Circular hole | 60 | 30 | 10 | 0 | Good |
| Example 4-24 | Acrylic-based coating film | Circular hole | 60 | 30 | 20 | 0 | Good |
| Example 4-25 | Acrylic-based coating film | Circular hole | 60 | 60 | 1 | 1 | Good |
| Example 4-26 | Acrylic-based coating film | Circular hole | 60 | 60 | 10 | 1 | Good |
| Example 4-27 | Acrylic-based coating film | Circular hole | 60 | 60 | 20 | 2 | Good |

As shown in Table 4, the test samples of Examples 4-1 to 4-27 were manufactured in the same manner as in Examples 1-1 to 1-27, except that an acrylic-based coating film (film molding) was used instead of polysulfone as a material of the base material 4.

The acrylic-based coating film was formed on a base member made of Noryl (registered trademark) resin. The outer shape of the base member was the same as that of the base material 4 except for the circular holes 5A.

The acrylic-based coating film was formed by electrostatic coating. The thickness of the acrylic-based coating film was 200 μm.

### Examples 5-1 to 5-27

The following Table 5 shows the base material configurations and evaluation results of Examples 5-1 to 5-27. Examples 5-1 to 5-27 are all Examples of the resin molding 6B of the first modified example.

TABLE 5

| | | | Recess | Recess | Recess | Cross- | Alcohol |
|---|---|---|---|---|---|---|---|
| | Base | Recess | depth | width | interval | cut | wiping |
| | material | shape | (μm) | (μm) | (μm) | test | test |
| Example 5-1 | Acrylic-based coating film | Square hole | 5 | 10 | 1 | 1 | Good |
| Example 5-2 | Acrylic-based coating film | Square hole | 5 | 10 | 10 | 0 | Good |
| Example 5-3 | Acrylic-based coating film | Square hole | 5 | 10 | 20 | 1 | Good |
| Example 5-4 | Acrylic-based coating film | Square hole | 5 | 30 | 1 | 0 | Good |
| Example 5-5 | Acrylic-based coating film | Square hole | 5 | 30 | 10 | 0 | Good |
| Example 5-6 | Acrylic-based coating film | Square hole | 5 | 30 | 20 | 1 | Good |
| Example 5-7 | Acrylic-based coating film | Square hole | 5 | 60 | 1 | 1 | Good |
| Example 5-8 | Acrylic-based coating film | Square hole | 5 | 60 | 10 | 1 | Good |
| Example 5-9 | Acrylic-based coating film | Square hole | 5 | 60 | 20 | 2 | Good |
| Example 5-10 | Acrylic-based coating film | Square hole | 30 | 10 | 1 | 0 | Good |
| Example 5-11 | Acrylic-based coating film | Square hole | 30 | 10 | 10 | 0 | Good |
| Example 5-12 | Acrylic-based coating film | Square hole | 30 | 10 | 20 | 0 | Good |
| Example 5-13 | Acrylic-based coating film | Square hole | 30 | 30 | 1 | 0 | Good |
| Example 5-14 | Acrylic-based coating film | Square hole | 30 | 30 | 10 | 0 | Good |
| Example 5-15 | Acrylic-based coating film | Square hole | 30 | 30 | 20 | 0 | Good |
| Example 5-16 | Acrylic-based coating film | Square hole | 30 | 60 | 1 | 1 | Good |
| Example 5-17 | Acrylic-based coating film | Square hole | 30 | 60 | 10 | 1 | Good |
| Example 5-18 | Acrylic-based coating film | Square hole | 30 | 60 | 20 | 1 | Good |
| Example 5-19 | Acrylic-based coating film | Square hole | 60 | 10 | 1 | 0 | Good |
| Example 5-20 | Acrylic-based coating film | Square hole | 60 | 10 | 10 | 0 | Good |
| Example 5-21 | Acrylic-based coating film | Square hole | 60 | 10 | 20 | 0 | Good |
| Example 5-22 | Acrylic-based coating film | Square hole | 60 | 30 | 1 | 0 | Good |
| Example 5-23 | Acrylic-based coating film | Square hole | 60 | 30 | 10 | 0 | Good |
| Example 5-24 | Acrylic-based coating film | Square hole | 60 | 30 | 20 | 0 | Good |
| Example 5-25 | Acrylic-based coating film | Square hole | 60 | 60 | 1 | 2 | Good |
| Example 5-26 | Acrylic-based coating film | Square hole | 60 | 60 | 10 | 2 | Good |
| Example 5-27 | Acrylic-based coating film | Square hole | 60 | 60 | 20 | 2 | Good |

As shown in Table 5, the test samples of Examples 5-1 to 5-27 were manufactured in the same manner as in Examples 2-1 to 2-27, except that an acrylic-based coating film (film molding) was used instead of polysulfone as the material of the base material 4. The acrylic-based coating film was formed in the same manner as in Examples 4-1 to 4-27.

### Examples 6-1 to 6-27

The following Table 6 shows the base material configurations and evaluation results of Examples 6-1 to 6-27.

Examples 6-1 to 6-27 are all examples of the resin molding 6C of the second modified example.

TABLE 6

| | | | Recess | Recess | Recess | Cross- | Alcohol |
|---|---|---|---|---|---|---|---|
| | Base | Recess | depth | width | interval | cut | wiping |
| | material | shape | (μm) | (μm) | (μm) | test | test |
| Example 6-1 | Acrylic-based coating film | Linear groove | 5 | 10 | 1 | 2 | Good |
| Example 6-2 | Acrylic-based coating film | Linear groove | 5 | 10 | 10 | 2 | Good |
| Example 6-3 | Acrylic-based coating film | Linear groove | 5 | 10 | 20 | 1 | Good |
| Example 6-4 | Acrylic-based coating film | Linear groove | 5 | 30 | 1 | 0 | Good |
| Example 6-5 | Acrylic-based coating film | Linear groove | 5 | 30 | 10 | 0 | Good |
| Example 6-6 | Acrylic-based coating film | Linear groove | 5 | 30 | 20 | 0 | Good |
| Example 6-7 | Acrylic-based coating film | Linear groove | 5 | 60 | 1 | 2 | Good |
| Example 6-8 | Acrylic-based coating film | Linear groove | 5 | 60 | 10 | 2 | Good |
| Example 6-9 | Acrylic-based coating film | Linear groove | 5 | 60 | 20 | 2 | Good |
| Example 6-10 | Acrylic-based coating film | Linear groove | 30 | 10 | 1 | 0 | Good |
| Example 6-11 | Acrylic-based coating film | Linear groove | 30 | 10 | 10 | 0 | Good |
| Example 6-12 | Acrylic-based coating film | Linear groove | 30 | 10 | 20 | 0 | Good |
| Example 6-13 | Acrylic-based coating film | Linear groove | 30 | 30 | 1 | 1 | Good |
| Example 6-14 | Acrylic-based coating film | Linear groove | 30 | 30 | 10 | 1 | Good |
| Example 6-15 | Acrylic-based coating film | Linear groove | 30 | 30 | 20 | 1 | Good |
| Example 6-16 | Acrylic-based coating film | Linear groove | 30 | 60 | 1 | 1 | Good |
| Example 6-17 | Acrylic-based coating film | Linear groove | 30 | 60 | 10 | 2 | Good |
| Example 6-18 | Acrylic-based coating film | Linear groove | 30 | 60 | 20 | 2 | Good |
| Example 6-19 | Acrylic-based coating film | Linear groove | 60 | 10 | 1 | 2 | Good |
| Example 6-20 | Acrylic-based coating film | Linear groove | 60 | 10 | 10 | 2 | Good |
| Example 6-21 | Acrylic-based coating film | Linear groove | 60 | 10 | 20 | 2 | Good |
| Example 6-22 | Acrylic-based coating film | Linear groove | 60 | 30 | 1 | 1 | Good |
| Example 6-23 | Acrylic-based coating film | Linear groove | 60 | 30 | 10 | 1 | Good |
| Example 6-24 | Acrylic-based coating film | Linear groove | 60 | 30 | 20 | 1 | Good |
| Example 6-25 | Acrylic-based coating film | Linear groove | 60 | 60 | 1 | 2 | Good |
| Example 6-26 | Acrylic-based coating film | Linear groove | 60 | 60 | 10 | 2 | Good |
| Example 6-27 | Acrylic-based coating film | Linear groove | 60 | 60 | 20 | 2 | Good |

As shown in Table 6, the test samples of Examples 6-1 to 6-27 were manufactured in the same manner as in Examples 3-1 to 3-27, except that an acrylic-based coating film (film molding) was used instead of polysulfone as the material of the base material 4. The acrylic-based coating film was formed in the same manner as in Examples 4-1 to 4-27.

### Examples 7-1 to 7-27

The following Table 7 shows the base material configurations and evaluation results of Examples 7-1 to 7-27. Examples 7-1 to 7-27 are all Examples of the resin molding 6A of the embodiment.

TABLE 7

| | | | Recess | Recess | Recess | Cross- | Alcohol |
|---|---|---|---|---|---|---|---|
| | Base | Recess | depth | width | interval | cut | wiping |
| | material | shape | (μm) | (μm) | (μm) | test | test |
| Example 7-1 | Polyphenylsulfide | Circular hole | 5 | 10 | 1 | 2 | Good |
| Example 7-2 | Polyphenylsulfide | Circular hole | 5 | 10 | 10 | 1 | Good |
| Example 7-3 | Polyphenylsulfide | Circular hole | 5 | 10 | 20 | 1 | Good |
| Example 7-4 | Polyphenylsulfide | Circular hole | 5 | 30 | 1 | 0 | Good |
| Example 7-5 | Polyphenylsulfide | Circular hole | 5 | 30 | 10 | 0 | Good |
| Example 7-6 | Polyphenylsulfide | Circular hole | 5 | 30 | 20 | 0 | Good |
| Example 7-7 | Polyphenylsulfide | Circular hole | 5 | 60 | 1 | 1 | Good |
| Example 7-8 | Polyphenylsulfide | Circular hole | 5 | 60 | 10 | 1 | Good |
| Example 7-9 | Polyphenylsulfide | Circular hole | 5 | 60 | 20 | 2 | Good |
| Example 7-10 | Polyphenylsulfide | Circular hole | 30 | 10 | 1 | 0 | Good |
| Example 7-11 | Polyphenylsulfide | Circular hole | 30 | 10 | 10 | 0 | Good |
| Example 7-12 | Polyphenylsulfide | Circular hole | 30 | 10 | 20 | 0 | Good |
| Example 7-13 | Polyphenylsulfide | Circular hole | 30 | 30 | 1 | 0 | Good |
| Example 7-14 | Polyphenylsulfide | Circular hole | 30 | 30 | 10 | 0 | Good |
| Example 7-15 | Polyphenylsulfide | Circular hole | 30 | 30 | 20 | 0 | Good |
| Example 7-16 | Polyphenylsulfide | Circular hole | 30 | 60 | 1 | 0 | Good |
| Example 7-17 | Polyphenylsulfide | Circular hole | 30 | 60 | 10 | 1 | Good |
| Example 7-18 | Polyphenylsulfide | Circular hole | 30 | 60 | 20 | 1 | Good |
| Example 7-19 | Polyphenylsulfide | Circular hole | 60 | 10 | 1 | 0 | Good |
| Example 7-20 | Polyphenylsulfide | Circular hole | 60 | 10 | 10 | 0 | Good |
| Example 7-21 | Polyphenylsulfide | Circular hole | 60 | 10 | 20 | 0 | Good |
| Example 7-22 | Polyphenylsulfide | Circular hole | 60 | 30 | 1 | 0 | Good |
| Example 7-23 | Polyphenylsulfide | Circular hole | 60 | 30 | 10 | 0 | Good |
| Example 7-24 | Polyphenylsulfide | Circular hole | 60 | 30 | 20 | 0 | Good |
| Example 7-25 | Polyphenylsulfide | Circular hole | 60 | 60 | 1 | 2 | Good |
| Example 7-26 | Polyphenylsulfide | Circular hole | 60 | 60 | 10 | 2 | Good |
| Example 7-27 | Polyphenylsulfide | Circular hole | 60 | 60 | 20 | 2 | Good |

As shown in Table 7, the test samples of Examples 7-1 to 7-27 were manufactured in the same manner as in Examples 1-1 to 1-27, except that polyphenylsulfide was used instead of polysulfone as the material of the base material 4.

### Examples 8-1 to 8-27

The following Table 8 shows the base material configurations and evaluation results of Examples 8-1 to 8-27. Examples 8-1 to 8-27 are all Examples of the resin molding 6B of the first modified example.

TABLE 8

| | | | Recess | Recess | Recess | Cross- | Alcohol |
|---|---|---|---|---|---|---|---|
| | Base | Recess | depth | width | interval | cut | wiping |
| | material | shape | (μm) | (μm) | (μm) | test | test |
| Example 8-1 | Polyphenylsulfide | Square hole | 5 | 10 | 1 | 2 | Good |
| Example 8-2 | Polyphenylsulfide | Square hole | 5 | 10 | 10 | 0 | Good |
| Example 8-3 | Polyphenylsulfide | Square hole | 5 | 10 | 20 | 0 | Good |
| Example 8-4 | Polyphenylsulfide | Square hole | 5 | 30 | 1 | 0 | Good |
| Example 8-5 | Polyphenylsulfide | Square hole | 5 | 30 | 10 | 0 | Good |
| Example 8-6 | Polyphenylsulfide | Square hole | 5 | 30 | 20 | 1 | Good |
| Example 8-7 | Polyphenylsulfide | Square hole | 5 | 60 | 1 | 1 | Good |
| Example 8-8 | Polyphenylsulfide | Square hole | 5 | 60 | 10 | 1 | Good |
| Example 8-9 | Polyphenylsulfide | Square hole | 5 | 60 | 20 | 2 | Good |
| Example 8-10 | Polyphenylsulfide | Square hole | 30 | 10 | 1 | 0 | Good |
| Example 8-11 | Polyphenylsulfide | Square hole | 30 | 10 | 10 | 0 | Good |
| Example 8-12 | Polyphenylsulfide | Square hole | 30 | 10 | 20 | 0 | Good |
| Example 8-13 | Polyphenylsulfide | Square hole | 30 | 30 | 1 | 0 | Good |
| Example 8-14 | Polyphenylsulfide | Square hole | 30 | 30 | 10 | 0 | Good |
| Example 8-15 | Polyphenylsulfide | Square hole | 30 | 30 | 20 | 1 | Good |
| Example 8-16 | Polyphenylsulfide | Square hole | 30 | 60 | 1 | 0 | Good |
| Example 8-17 | Polyphenylsulfide | Square hole | 30 | 60 | 10 | 0 | Good |
| Example 8-18 | Polyphenylsulfide | Square hole | 30 | 60 | 20 | 1 | Good |
| Example 8-19 | Polyphenylsulfide | Square hole | 60 | 10 | 1 | 0 | Good |
| Example 8-20 | Polyphenylsulfide | Square hole | 60 | 10 | 10 | 0 | Good |
| Example 8-21 | Polyphenylsulfide | Square hole | 60 | 10 | 20 | 0 | Good |
| Example 8-22 | Polyphenylsulfide | Square hole | 60 | 30 | 1 | 1 | Good |
| Example 8-23 | Polyphenylsulfide | Square hole | 60 | 30 | 10 | 1 | Good |
| Example 8-24 | Polyphenylsulfide | Square hole | 60 | 30 | 20 | 1 | Good |
| Example 8-25 | Polyphenylsulfide | Square hole | 60 | 60 | 1 | 2 | Good |
| Example 8-26 | Polyphenylsulfide | Square hole | 60 | 60 | 10 | 2 | Good |
| Example 8-27 | Polyphenylsulfide | Square hole | 60 | 60 | 20 | 2 | Good |

As shown in Table 8, the test samples of Examples 8-1 to 8-27 were manufactured in the same manner as in 2-1 to 2-27, except that polyphenylsulfide was used instead of polysulfone as the material of the base material 4.

### Examples 9-1 to 9-27

The following Table 9 shows the base material configurations and evaluation results of Examples 9-1 to 9-27. Examples 9-1 to 9-27 are all Examples of the resin molding 6C of the second modified example.

TABLE 9

| | | | Recess | Recess | Recess | Cross- | Alcohol |
|---|---|---|---|---|---|---|---|
| | Base | Recess | depth | width | interval | cut | wiping |
| | material | shape | (μm) | (μm) | (μm) | test | test |
| Example 9-1 | Polyphenylsulfide | Linear groove | 5 | 10 | 1 | 2 | Good |
| Example 9-2 | Polyphenylsulfide | Linear groove | 5 | 10 | 10 | 2 | Good |
| Example 9-3 | Polyphenylsulfide | Linear groove | 5 | 10 | 20 | 1 | Good |
| Example 9-4 | Polyphenylsulfide | Linear groove | 5 | 30 | 1 | 0 | Good |
| Example 9-5 | Polyphenylsulfide | Linear groove | 5 | 30 | 10 | 0 | Good |
| Example 9-6 | Polyphenylsulfide | Linear groove | 5 | 30 | 20 | 0 | Good |
| Example 9-7 | Polyphenylsulfide | Linear groove | 5 | 60 | 1 | 2 | Good |
| Example 9-8 | Polyphenylsulfide | Linear groove | 5 | 60 | 10 | 2 | Good |
| Example 9-9 | Polyphenylsulfide | Linear groove | 5 | 60 | 20 | 2 | Good |
| Example 9-10 | Polyphenylsulfide | Linear groove | 30 | 10 | 1 | 0 | Good |
| Example 9-11 | Polyphenylsulfide | Linear groove | 30 | 10 | 10 | 0 | Good |
| Example 9-12 | Polyphenylsulfide | Linear groove | 30 | 10 | 20 | 0 | Good |
| Example 9-13 | Polyphenylsulfide | Linear groove | 30 | 30 | 1 | 1 | Good |
| Example 9-14 | Polyphenylsulfide | Linear groove | 30 | 30 | 10 | 1 | Good |
| Example 9-15 | Polyphenylsulfide | Linear groove | 30 | 30 | 20 | 1 | Good |
| Example 9-16 | Polyphenylsulfide | Linear groove | 30 | 60 | 1 | 1 | Good |
| Example 9-17 | Polyphenylsulfide | Linear groove | 30 | 60 | 10 | 2 | Good |
| Example 9-18 | Polyphenylsulfide | Linear groove | 30 | 60 | 20 | 2 | Good |
| Example 9-19 | Polyphenylsulfide | Linear groove | 60 | 10 | 1 | 2 | Good |
| Example 9-20 | Polyphenylsulfide | Linear groove | 60 | 10 | 10 | 2 | Good |
| Example 9-21 | Polyphenylsulfide | Linear groove | 60 | 10 | 20 | 2 | Good |
| Example 9-22 | Polyphenylsulfide | Linear groove | 60 | 30 | 1 | 1 | Good |
| Example 9-23 | Polyphenylsulfide | Linear groove | 60 | 30 | 10 | 1 | Good |
| Example 9-24 | Polyphenylsulfide | Linear groove | 60 | 30 | 20 | 1 | Good |
| Example 9-25 | Polyphenylsulfide | Linear groove | 60 | 60 | 1 | 2 | Good |
| Example 9-26 | Polyphenylsulfide | Linear groove | 60 | 60 | 10 | 2 | Good |
| Example 9-27 | Polyphenylsulfide | Linear groove | 60 | 60 | 20 | 2 | Good |

As shown in Table 9, the test samples of Examples 6-1 to 6-27 were manufactured in the same manner as in Examples 3-1 to 3-27, except that polyphenylsulfide was used instead of polysulfone as the material of the base material 4.

### Comparative Examples

The following Table 10 shows the base material configurations and evaluation results of each of the Comparative Examples.

TABLE 10

| | | | Recess | Recess | Recess | Cross- | Alcohol |
|---|---|---|---|---|---|---|---|
| | Base | Recess | depth | width | interval | cut | wiping |
| | material | shape | (μm) | (μm) | (μm) | test | test |
| Comparative Example 1 | Polysulfone | None | — | — | — | 5 | NG |
| Comparative Example 2-1 | Polysulfone | Circular hole | 80 | 70 | 50 | 4 | NG |
| Comparative Example 2-2 | Polysulfone | Circular hole | 1 | 5 | 50 | 4 | NG |
| Comparative Example 3-1 | Polysulfone | Square hole | 80 | 70 | 50 | 4 | NG |
| Comparative Example 3-2 | Polysulfone | Square hole | 1 | 5 | 50 | 4 | NG |
| Comparative Example 4-1 | Polysulfone | Linear groove | 80 | 70 | 50 | 5 | NG |
| Comparative Example 4-2 | Polysulfone | Linear groove | 1 | 5 | 50 | 5 | NG |

As shown in Table 10, a test sample of the resin molding for a medical device of Comparative Example 1 was manufactured in the same manner as in Example 1-1 except that the circular holes 5A were not provided in the base material 4.

The test samples of the resin moldings for a medical device of Comparative Examples 2-1 and 2-2 were manufactured in the same manner as the test sample in Example 1-1, except that the size of the circular holes was different from that of the circular holes 5A of Example 1-1.

In the circular holes in Comparative Example 2-1, the recess depth, the recess width, and the recess interval were 80 μm, 70 μm, and 50 μm, respectively. In the circular holes in Comparative Example 2-2, the recess depth, the recess width, and the recess interval were 1 μm, 5 μm, and 50 μm, respectively.

The test samples of the resin moldings for a medical device of Comparative Examples 3-1 and 3-2 were manufactured in the same manner as the test sample in Example 1-2, except that the dimensions of the square holes were different from the square holes 5B of Example 1-2.

In the square holes in Comparative Example 3-1, the recess depth, recess width, and recess interval were 80 μm, 70 μm, and 50 μm, respectively. In the square holes in Comparative Example 3-2, the recess depth, the recess width, and the recess interval were 1 μm, 5 μm, and 50 μm, respectively.

The test samples of the resin moldings for a medical device of Comparative Examples 4-1 and 4-2 were manufactured in the same manner as the test sample in Examples 1-3, except that the dimensions of the linear grooves were different from the linear grooves 5C of Example 1-2.

In the linear grooves in Comparative Example 4-1, the recess depth, recess width, and recess interval were 80 μm, 70 μm, and 50 μm, respectively. In the linear grooves in Comparative Example 4-2, the recess depth, the recess width, and the recess interval were 1 μm, 5 μm, and 50 μm, respectively.

(Evaluation)

As shown in Table 1 to Table 10, a cross-cut test and an alcohol wiping test were performed as evaluations of each Example and each Comparative Example.

The cross-cut test was performed on the indicators of each Example and each Comparative Example in accordance with the cross-cut method of JIS K5600-5-6. The evaluation results were represented by classifications 0 to 5 according to the above JIS. The smaller the classification number, the better the adhesion.

In a case where the evaluation result was a classification of 0 to 2, the adhesion between the indicator and the base material was determined to be good. In a case where the evaluation result was a classification of 3 to 5, the adhesion between the indicator and the base material was determined to be poor.

In the alcohol wiping test, the surface of the test sample including the indicator was repeatedly wiped using gauze including ethanol. As the gauze, 5 cm×5 cm of sterile Kpain (registered trademark) (trade name; manufactured by Kawamoto Corporation) was used. For the number of times of wiping, wiping one time with one reciprocation was performed 3,000 times on the indicator.

In a case where the indicator 3 was not peeled off even after wiping 1000 times or more, the adhesion between the indicator and the base material was determined to be good (described as “good” in each table). In a case where the indicator 3 was peeled off after wiping less than 1000 times, the adhesion between the indicator and the base material was determined to be poor (described in each table as “NG” (no good)).

As shown in Table 1 to Table 9, in all of the Examples described above, the evaluation results of the cross cut test were any one of classifications 0, 1, and 2. Furthermore, in all the Examples described above, the evaluation results of the alcohol wiping test were good.

Accordingly, in all the Examples described above, the adhesion between the indicator 3 and the base material 4 was good.

On the other hand, in all the Comparative Examples described above, the evaluation results of the cross-cut test were either of classifications 4 and 5. Furthermore, in all the Comparative Examples described above, the evaluation results of the alcohol wiping test were poor.

Accordingly, in all the Comparative Examples described above, the adhesion between the indicator and the base material was poor.

For example, in Comparative Example 1, it is considered that the adhesion was poor due to the fact that no recesses were provided in the base material.

For example, in Comparative Examples 2-1, 3-1, and 4-1, it is considered that the adhesion was poor due to the fact that at least the recess width exceeded 60 μm.

For example, in Comparative Examples 2-2, 3-2, and 4-2, it is considered that the adhesion was poor due to the fact that at least the recess width was less than 10 μm.

Although preferable embodiments of the present invention were described above together with the respective modified examples and Examples, the present invention is not limited to the embodiments, the modified examples, or the Examples. Additions, omissions, substitutions, and other modifications of the configuration are possible in a range not departing from the spirit of the present invention.

The present invention is not limited by the above description, and is limited only by the appended claims.

According to the embodiment and each modified example described above, it is possible to provide a resin molding for a medical device, which is able to be manufactured quickly and easily and which is able to improve adhesion between an indicator and a base material, and a medical device.

What is claimed is:

1. A resin molding for a medical device comprising:
a base material made of resin; and
an indicator including a cured product of UV-curable ink,
wherein an uneven structure is formed on at least a part of a surface of the base material,
the uneven structure includes a plurality of recesses,
the plurality of recesses each have a width of 10 μm or more and 60 μm or less, and
the indicator is formed on the uneven structure.

2. The resin molding for a medical device according to claim 1,
wherein a depth of each of the plurality of recesses is 5 μm or more and 60 μm or less.

3. The resin molding for a medical device according to claim 1,
wherein an interval between recesses adjacent to each other among the plurality of recesses is 1 μm or more and 30 μm or less.

4. The resin molding for a medical device according to claim 1,
wherein an arrangement of the plurality of recesses is periodic.

5. The resin molding for a medical device according to claim 1,
wherein the plurality of recesses include at least one of holes and grooves.

6. A medical device comprising a resin molding for a medical device, the resin molding including:
a base material made of resin; and
an indicator including a cured product of UV-curable ink,
wherein an uneven structure is formed on at least a part of a surface of the base material,
the uneven structure includes a plurality of recesses,
the plurality of recesses each have a width of 10 μm or more and 60 μm or less, and
the indicator is formed on the uneven structure.
